(19) United States
(12) Patent Application Publication
(10) Pub. No.: US 2013/0150936 A1
(43) Pub. Date: Jun. 13, 2013

(54) NERVE STIMULATING DEVICE AND NERVE STIMULATING SYSTEM

(71) Applicant: OLYMPUS CORPORATION, Tokyo (JP)

(72) Inventor: Hiroshi TAKAHASHI, Tokyo (JP)

(73) Assignee: OLYMPUS CORPORATION, Tokyo (JP)

(21) Appl. No.: 13/711,013

(22) Filed: Dec. 11, 2012

(30) Foreign Application Priority Data
Dec. 13, 2011 (JP) 2011-272678

(51) Int. Cl. A61N 1/05

(52) U.S. Cl. CPC A61N 1/0507; A61N 1/0517
USPC 607/116

(57) ABSTRACT

Provided is a nerve stimulating device with which unwanted bacteria, etc. are prevented from entering a body, which serves to reduce the risk of infection. The nerve stimulating device includes a stent that is indwelled inside the body, a receiving coil that is provided in the stent and that receives energy from outside the body, and stimulating electrodes that are provided in the stent and that convert the energy received by the receiving coil into electric power at the timing at which the receiving coil receives the energy and passively output the electric power as nerve stimulating pulses.

### CROSS-REFERENCE TO RELATED APPLICATIONS

This application is based on Japanese Patent Application No. 2011-272678, the contents of which are incorporated herein by reference.

### BACKGROUND OF THE INVENTION

1. Field of the Invention

The present invention relates to a nerve stimulating device for stimulating a nerve and to a nerve stimulating system including the same.

2. Description of Related Art

Nerve stimulating devices of the type that includes electrodes inserted into the pancreatic duct of a subject and a power feeding unit for feeding electric power to the electrodes in order to electrically stimulate the pancreas are known (e.g., see the specification of United States Patent Application Publication No. 2006/0004422).

In the nerve stimulating device disclosed in the specification of United States Patent Application Publication No. 2006/0004422, the power feeding unit is placed outside the body, and it is connected via a lead wire to the electrodes placed inside the body. Thus, since the lead wire penetrates the body surface, unwanted bacteria, etc. may enter the body via the penetrating region, causing infection.

### BRIEF SUMMARY OF THE INVENTION

The present invention provides a nerve stimulating device for stimulating a nerve, with which unwanted bacteria, etc. can be prevented from entering a body, which serves to reduce the risk of infection, and to provide a nerve stimulating system including the same.

The present invention employs the following solutions.

A first aspect of the present invention is a nerve stimulating device including a main unit that is indwelled inside a body; a receiving unit that is provided in the main unit and that receives energy from outside the body; and a stimulating-pulse outputting unit that is provided in the main unit and that converts the energy received by the receiving unit into electric power at the timing at which the receiving unit receives the energy and passively outputs the electric power as nerve stimulating pulses.

According to the first aspect of the present invention, with the main unit indwelled inside the body, the receiving unit receives energy from outside the body, and the stimulating-pulse outputting unit converts the received energy into electric power at the timing at which the receiving unit receives the energy and passively outputs the electric power as nerve stimulating pulses.

More specifically, for example, it is possible to promote insulin secretion by indwelling the nerve stimulating device according to the first aspect of the present invention in a bile duct and stimulating an efferent vagus nerve and pancreatic β cells (insulin-secreting cells) in the vicinity of the bile duct. Furthermore, by stimulating an afferent vagus nerve in the vicinity of a bile duct with the nerve stimulating device according to the present invention, it is possible to increase the number of pancreatic β cells, thereby increasing insulin secretion.

In this case, with the nerve stimulating device according to the first aspect of the present invention, it is possible to wirelessly receive energy from outside the body, convert the energy into electric power, and stimulate nerves. Thus, a lead wire for connecting the main unit inside the body and a power feeding unit outside the body, as well as a penetrating region where the lead wire penetrates, can be eliminated. This serves to prevent unwanted bacteria, etc. from entering the body from the penetrating region on the body surface, serving to reduce the risk of infection.

In the above aspect, the nerve stimulating device may further include a retaining unit that is guided out from the distal end of an inserted portion of an endoscope and that retains the main unit at a designated position in the body.

In this way, with the inserted portion of the endoscope inserted into a body cavity, it is possible to guide out the nerve stimulating device according to the present invention from the distal end (channel) thereof and to place it at the designated position in the body cavity. Then, it is possible to retain the main unit at the designated position in the body by using the retaining unit. Accordingly, it is possible to indwell the nerve stimulating device according to the present invention at the desired position in the body cavity without performing surgery. This serves to reduce the level of invasiveness, thereby alleviating the burden on the patient.

In the above aspect, the retaining unit may be a stent that is inserted into a lumen.

In this way, when guiding out the nerve stimulating device according to the present invention from the inserted portion of the endoscope, it is possible to contract the stent, making the main unit compact. This facilitates the task of guiding out the nerve stimulating device. Furthermore, since the stent is a cylindrical component that can be expanded inside the body, it is possible to prevent interference with the flows of body fluids when the stent is indwelled in the body cavity.

In the above aspect, the retaining unit may retain the main unit in the vicinity of the gastric cardia of the esophagus, in the duodenum, or in a bile duct, close to where a vagus nerve runs.

In this way, it is possible to effectively stimulate the vagus nerve running in the vicinity of the gastric cardia of the esophagus, or in the vicinity of the duodenum or the bile duct, serving to increase insulin secretion from the pancreas.

In the above aspect, the retaining unit may be composed of a biodegradable material.

In this way, after the main unit of the device is indwelled inside the body with the retaining unit for a certain period, the retaining unit loses its retaining force, is detached, and is finally discharged outside of the body. Thus, it is possible to discharge the main unit outside of the body after a certain period instead of permanently indwelling it inside the body, so that an operation for removing the main unit is not needed.

In the above aspect, the stimulating-pulse outputting unit may stimulate an afferent vagus nerve that is located distal to the celiac ganglia.

By stimulating the vagus nerve distal to the celiac ganglia, it is possible to stimulate only the target nerve. This serves to effectively increase insulin secretion from the pancreas while preventing side effects caused by stimulating other nerves. Furthermore, by stimulating an afferent vagus nerve, it is possible to increase the number of pancreatic β cells while preventing deterioration of the function of the pancreatic β cells in the mid to long term. This serves to effectively increase insulin secretion from the pancreas.

In the above aspect, the receiving unit may receive AC electric power, and the stimulating-pulse outputting unit may include a rectifying unit that rectifies the AC electric power received by the receiving unit and output the electric power rectified by the rectifying unit in the form of pulses.

In this way, the AC electric power received by the receiving unit is rectified by the rectifying unit, and the rectified electric power is output as pulses (nerve stimulating pulses). This serves to promote insulin secretion from the pancreas.

In the above aspect, the stimulating-pulse outputting unit may include a resonant circuit that resonates with the AC electric power received by the receiving unit.

In this case, by causing the resonant circuit to resonate with the AC electric power received by the receiving unit, it is possible to stimulate a nerve with stimulating pulses having an amplified amplitude (i.e., output). This serves to effectively promote insulin secretion from the pancreas.

In the above aspect, the nerve stimulating device may include a magnetostrictive component exhibiting a magnetostrictive effect, the receiving unit may receive vibration from outside the body, and the stimulating-pulse outputting unit may output electric power generated by the magnetostrictive effect of the magnetostrictive component as nerve stimulating pulses.

In this way, the receiving unit receives vibrations from outside the body, which enables the stimulating-pulse outputting unit to output the electric power generated by the magnetostrictive effect of the magnetostrictive component as nerve stimulating pulses.

A second aspect of the present invention is a nerve stimulating system including the above nerve stimulating device and an energy supplying unit that is placed outside the body and that supplies energy to the receiving unit.

With this nerve stimulating system, since the above-described nerve stimulating device is provided, it is possible to supply energy from the energy supplying unit placed outside the body to the receiving unit placed inside the body, enabling the stimulating-pulse outputting unit to passively output nerve stimulating pulses. This serves to increase insulin secretion from the pancreas. Furthermore, it is possible to prevent unwanted bacteria, etc. from entering the body via a penetrating region on the body surface, which serves to reduce the risk of infection.

A third aspect of the present invention is a nerve stimulating method wherein stimulating electrodes for stimulating a nerve are indwelled in a body cavity and wherein the stimulating electrodes receive energy from outside a body, convert the received energy into electric power at the timing at which the energy is received, and passively output the electric power as nerve stimulating pulses.

In the above aspect, an inserted portion of an endoscope may be inserted into the body cavity, and the stimulating electrodes may be guided out from the distal end of the inserted portion and indwelled in the body cavity.

In the above aspect, a retaining unit that retains the stimulating electrodes at a designated position in the body may be provided, and the retaining unit may be expanded in the body cavity to retain the stimulating electrodes at the designated position in the body cavity.

In the above aspect, the retaining unit may be a stent that is inserted into a lumen.

In the above aspect, the retaining unit may retain the stimulating electrodes in the vicinity of the gastric cardia of the esophagus, in the duodenum, or in a bile duct, close to where a vagus nerve runs.

In the above aspect, the stimulating electrodes may stimulate an afferent vagus nerve that is located distal to the celiac ganglia.

According to the present invention, an advantage is afforded in that unwanted bacteria, etc. can be prevented from entering the body, which serves to reduce the risk of infection.

### BRIEF DESCRIPTION OF THE SEVERAL VIEWS OF THE DRAWINGS

FIG. 1 is a diagram showing the overall configuration of a nerve stimulating system according to a first embodiment of the present invention.

FIG. 2 is a side view of a nerve stimulating device according to the first embodiment.

FIG. 3 is an illustration showing the overall construction of the nerve stimulating device in FIG. 2.

FIG. 4 is a functional block diagram of the nerve stimulating device in FIG. 2.

FIG. 5 is a diagram schematically showing the configuration of an energy supplying unit according to the first embodiment.

FIG. 6 is a functional block diagram of the energy supplying unit in FIG. 5.

FIG. 7 is a timing chart of stimulating pulses of the nerve stimulating system according to the first embodiment, in which part (a) shows stimulating pulses at the nerve stimulating device and part (b) shows stimulating pulses at the energy supplying unit.

FIG. 8 is an illustration showing the procedure for indwelling the nerve stimulating device in FIG. 1 in a body cavity by using an endoscope, where part (a) shows a state before expanding a stent, part (b) shows a state in the middle of expansion of the stent, and part (c) shows a state after expansion of the stent.

FIG. 9 is an illustration for explaining the positions of the vagus nerve and a bile duct.

FIG. 10 is an illustration for explaining placement (orientation) of the nerve stimulating system in FIG. 1 inside a body.

FIG. 11 is an illustration for explaining placement of the nerve stimulating system in FIG. 1 during nerve stimulation.

FIG. 12 is an illustration showing the overall construction of a nerve stimulating device according to a second embodiment.

FIG. 13 is a functional block diagram of the nerve stimulating device in FIG. 12.

FIG. 14 is a diagram showing the overall configuration of an energy supplying unit according to the second embodiment.

FIG. 15 is a functional block diagram of the energy supplying unit in FIG. 14.

FIG. 16 is a timing chart of stimulating pulses of a nerve stimulating system according to the second embodiment, in which part (a) shows stimulating pulses at the nerve stimulating device and part (b) shows stimulating pulses at the energy supplying unit.

FIG. 17 is an illustration showing the overall construction of a nerve stimulating device according to a third embodiment.

FIG. 18 is an illustration for explaining a magnetostrictive effect.

FIG. 19 is a diagram showing the overall configuration of an energy supplying unit according to the third embodiment.

### DETAILED DESCRIPTION OF THE INVENTION

### First Embodiment

A nerve stimulating device 10 and a nerve stimulating system 1 including the same according to a first embodiment of the present invention will be described below with reference to the drawings.

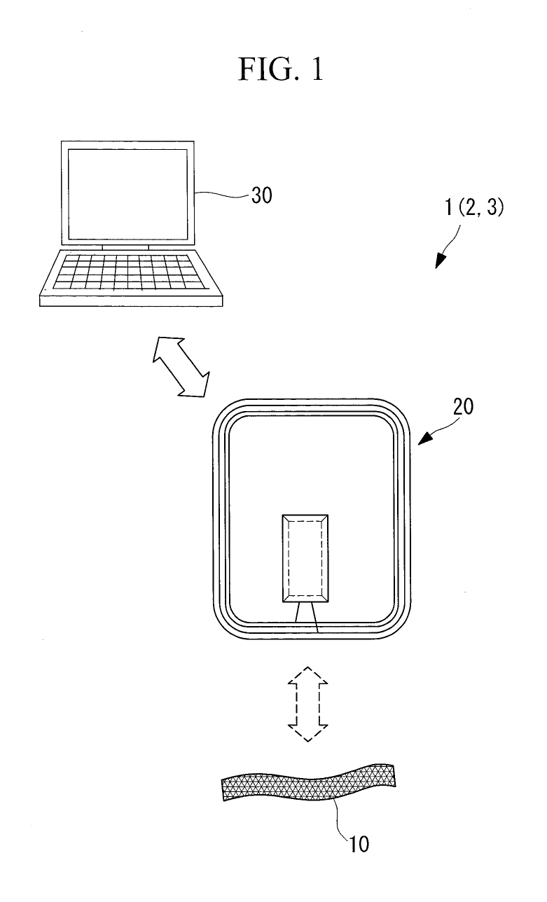

As shown in FIG. 1, the nerve stimulating system 1 according to this embodiment includes the nerve stimulating device 10, which is indwelled in a body cavity, an energy supplying unit 20 that is placed outside the body and that supplies energy to the nerve stimulating device 10, and a control device 30 that controls these components.

The nerve stimulating device 10 and the energy supplying unit 20 are electromagnetically coupled with each other in such a manner as to allow the supply of energy, such as electric power, as will be described later.

The energy supplying unit 20 and the control device 30 are configured to perform wireless communication with each other.

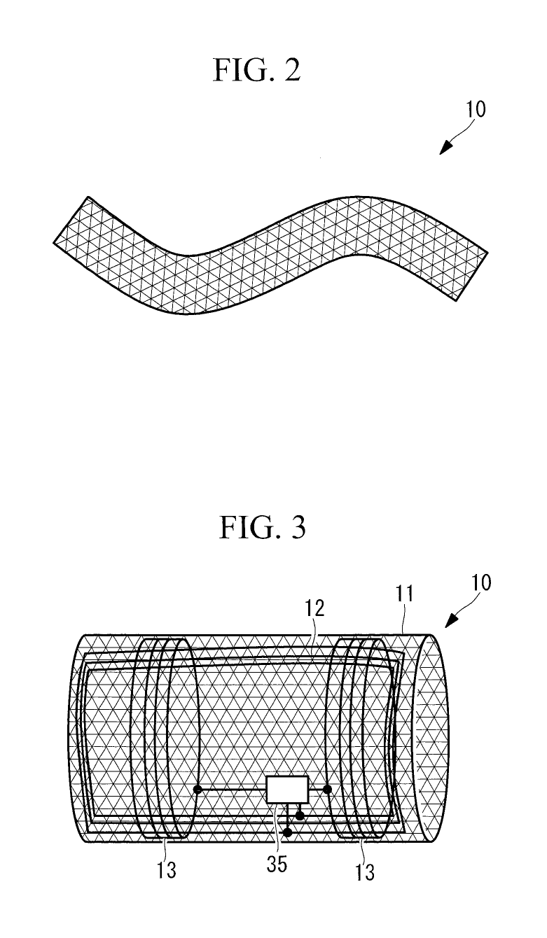

As shown in FIG. 2, the main unit of the nerve stimulating device 10 is constructed in the form of a stent. Here, the stent refers to a mesh-like cylinder constructed of, for example, a metal, such as stainless steel, which can be contracted, expanded, and bent. More specifically, for example, the stent is a medical instrument that is inserted into a coronary artery, which is a blood vessel of the heart, in the event of angina pectoris or myocardial infarction and is used to widen the coronary artery in a region where treatment is to be performed.

As shown in FIG. 3, the nerve stimulating device 10 includes a cylindrical stent (main unit, retaining unit) 11 like that described above, a receiving coil (receiving unit) 12 provided in the stent 11, and stimulating electrodes (stimulating-pulse outputting unit) 13 provided in the stent 11.

The stent 11 having the construction described above is inserted into a body cavity, such as a bile duct, and is expanded at a desired position where treatment is to be performed, whereby the nerve stimulating device 10 is retained at the desired position. That is, the stent 11 functions as a retaining unit that retains the nerve stimulating device 10.

The receiving coil 12 is a metallic coil disposed along the longitudinal direction and circumferential direction of the cylindrical stent 11. The receiving coil 12 is configured to receive energy (electric power in this embodiment) from the energy supplying unit 20 placed outside the body.

The stimulating electrodes 13 are metallic coils disposed along the circumferential direction of the cylindrical stent 11. The stimulating electrodes 13 are disposed to form a pair in the axial direction (longitudinal direction) of the stent 11. The stimulating electrodes 13 are electrically connected to the receiving coil 12 via a rectifying unit 35.

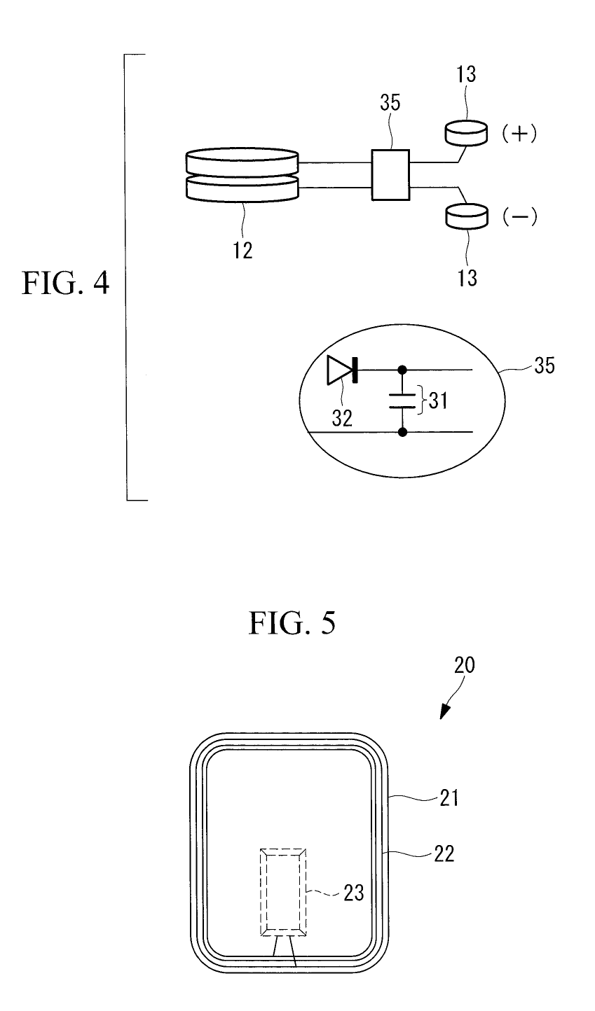

As shown in FIG. 4, the rectifying unit 35 includes a diode 32 that rectifies AC electric power received by the receiving coil 12 and a capacitor 31 that stores the rectified electric power. The rectifying unit 35 may be configured to perform either half-wave rectification or full-wave rectification.

The thus-configured stimulating electrodes 13 convert the energy received by the receiving coil 12 into electric power at the timing at which the receiving coil 12 receives the energy from the energy supplying unit 20 placed outside the body and passively output the electric power as nerve stimulating pulses.

With the inserted portion of an endoscope inserted into a body cavity, the nerve stimulating device 10 configured as described above is guided out from the distal end (channel) of the inserted portion of the endoscope to indwell the nerve stimulating device 10 at a designated position in the body cavity. The operation carried out at this time will be described later in detail.

As shown in FIG. 5, the energy supplying unit 20 includes an adhesive sheet 21 that is attached to the body surface, a coil antenna 22 that feeds electric power wirelessly to the receiving coil 12 of the nerve stimulating device 10 placed inside the body cavity, and a generator 23 that supplies electric power to the coil antenna 22.

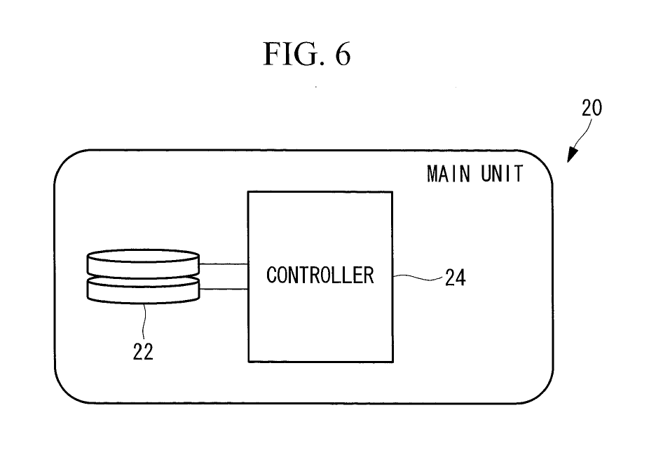

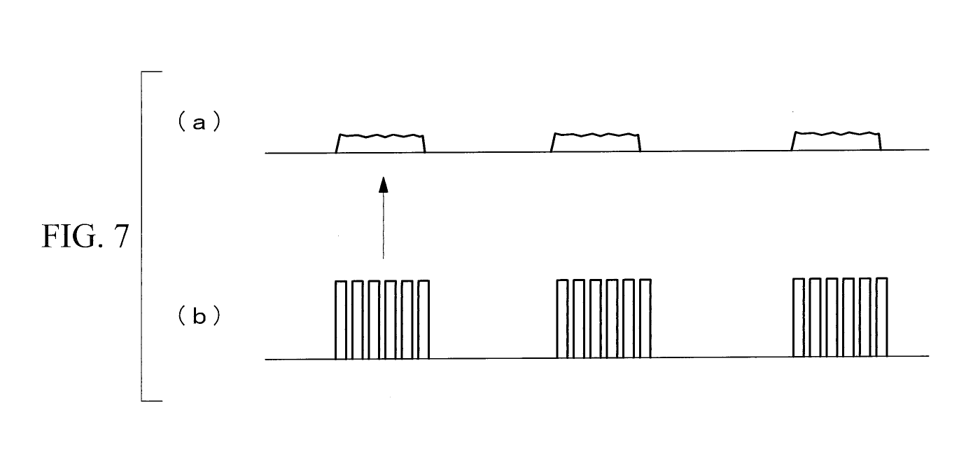

Furthermore, as shown in FIG. 6, the energy supplying unit 20 includes a controller 24 that controls the generator 23 on the basis of a wireless communication instruction from the control device 30. The generator 23 downloads an operating program for a stimulating pattern in advance on the basis of the wireless communication instruction from the control device 30, and the controller 24 feeds pulsed electric power output to the nerve stimulating device 10 according to the operating program.

As shown in part (b) of FIG. 7, the controller 24 controls the generator 23 so that the generator 23 outputs pulsed electric power to the coil antenna 22. Furthermore, the controller 24 also controls the DC pulse rate and the output level of the pulsed electric power output from the generator 23.

Thus, the receiving coil 12 of the nerve stimulating device 10 receives the pulsed electric power from the energy supplying unit 20. Accordingly, the stimulating electrodes 13 of the nerve stimulating device 10 output nerve stimulating pulses at the timing at which the receiving coil 12 receives energy from the energy supplying unit 20 placed outside the body, as shown in part (a) of FIG. 7.

The procedure for indwelling the nerve stimulating device 10 in a body cavity by using an endoscope in the thus-configured nerve stimulating system 1 according to this embodiment will be described below.

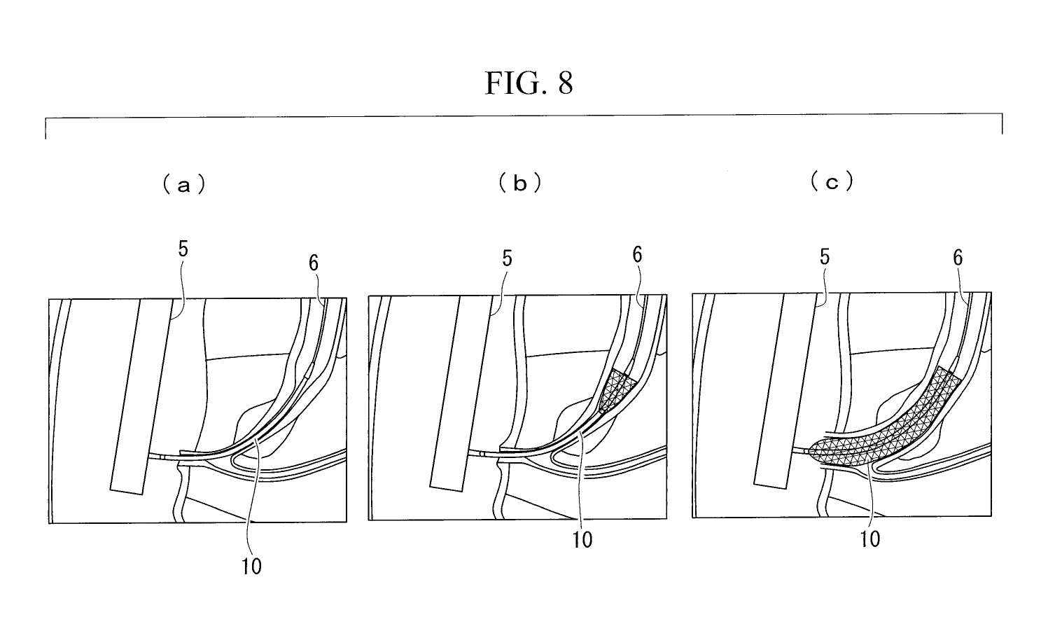

First, as shown in part (a) of FIG. 8, the inserted portion of a duodenal endoscope 5 is inserted into the duodenum via the esophagus and the stomach, and a delivery catheter equipped with the nerve stimulating device 10 (stent 11) at the distal end thereof is inserted to a stimulating region along a guide wire 6 inserted through a forceps insertion channel of the endoscope 5 from the duodenal papilla to a bile duct.

Then, as shown in part (b) of FIG. 8, while checking the position where the nerve stimulating device 10 (stent 11) is indwelled in the body cavity by using X rays, the nerve stimulating device 10 (stent 11) is placed at a desired position in the bile duct by operating the proximal end of the delivery catheter, and the stent 11 is expanded outward in the radial direction.

Thus, as shown in part (c) of FIG. 8, the expanded stent 11 is urged against the inner wall of the bile duct, and the nerve stimulating device 10 is indwelled at the desired position in the bile duct by this urging force.

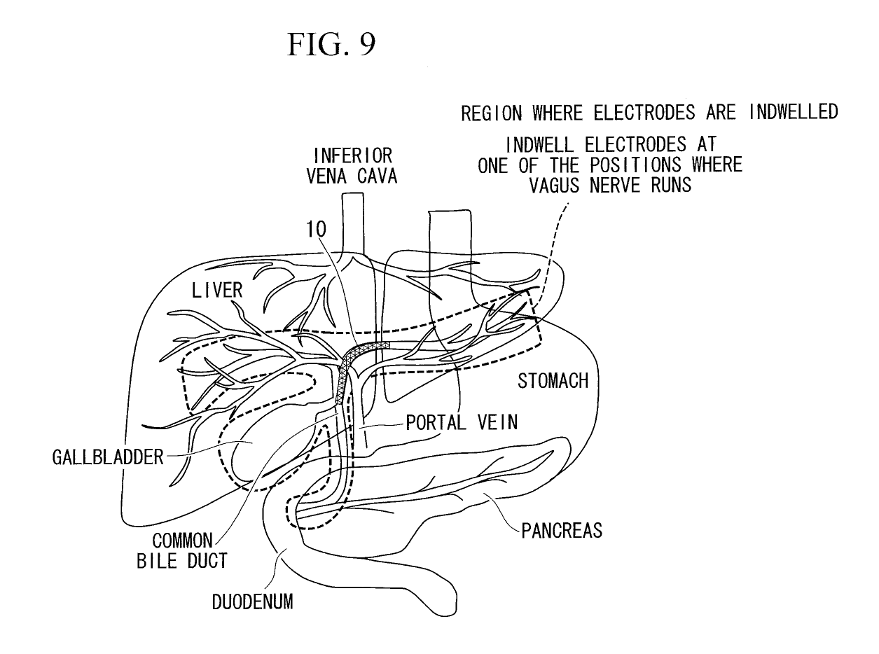

Here, the vagus nerve runs along the common bile duct, as shown in FIG. 9. Thus, as described earlier, it is possible to stimulate the vagus nerve effectively by indwelling the nerve stimulating device 10 according to this embodiment in the common bile duct, close to where the vagus nerve runs, and causing the nerve stimulating device 10 to output stimulating pulses.

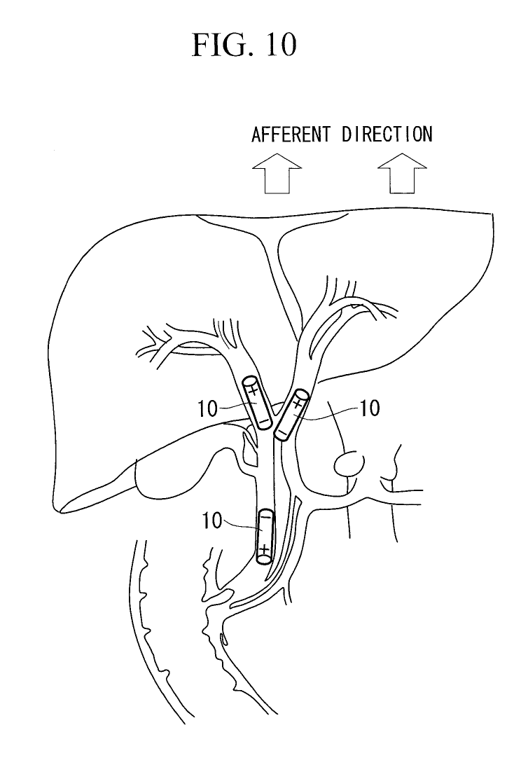

In this case, it is desired that the nerve stimulating device 10 according to this embodiment be placed so that an afferent vagus nerve will be stimulated with stimulating pulses, as shown in FIG. 10. More specifically, as shown in FIG. 10, the nerve stimulating device 10 is placed with the positive electrode at the organ (e.g., the pancreas) side and the negative electrode at the brain side. In this way, a current flows from the organ side to the brain side, making it possible to stimulate the vagus nerve in the afferent direction. Although an example where three stents 11 (nerve stimulating devices 10) are indwelled is shown in FIG. 10, basically, a single stent 11 (nerve stimulating device 10) may be indwelled. Furthermore, the stents 11 should be placed with different electrode polarities depending on the indwelling positions thereof so that afferent stimuli can be given.

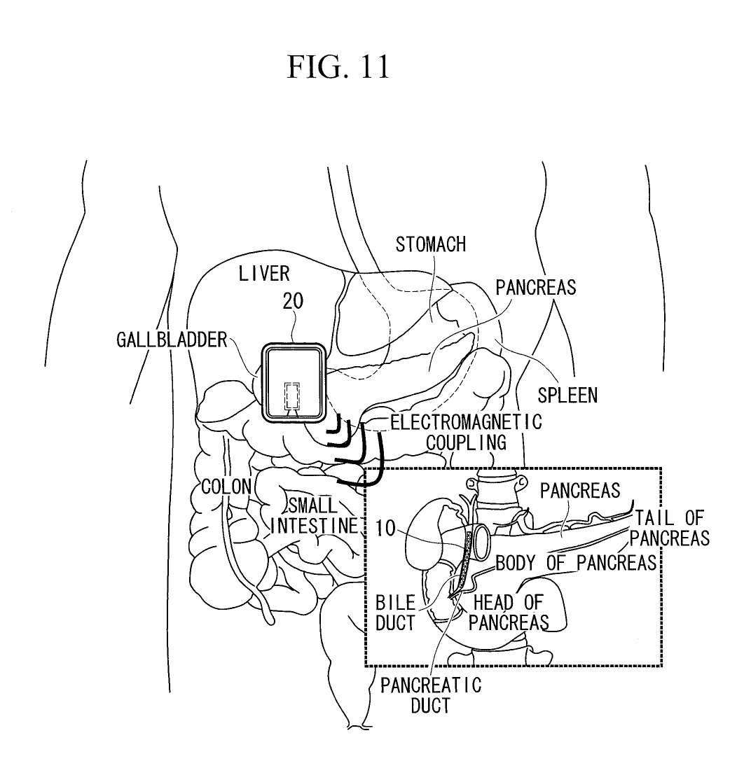

With the nerve stimulating device 10 indwelled at the desired position in the bile duct as described above, the energy supplying unit 20 is attached to the body surface in the vicinity of the bile duct, as shown in FIG. 11. Then, the generator 23 of the energy supplying unit 20 is activated, whereby the coil antenna 22 of the energy supplying unit 20 and the receiving coil 12 of the nerve stimulating device 10 are electromagnetically coupled.

In this way, while controlling the DC pulse rate and the output level at the energy supplying unit 20 side via the coil antenna 22 from the generator 23 of the energy supplying unit 20 attached to the body surface, stimulating pulses are supplied wirelessly to the receiving coil 12 of the nerve stimulating device 10 indwelled in the bile duct.

Now, problems in stimulating the vagus nerve in the vicinity of the pancreas by using existing nerve stimulating devices will be described.

In the case of one type of existing nerve stimulating device (e.g., Japanese Translation of PCT International Application, Publication No. Hei 7-503865), electrodes are surgically embedded in the vicinity of the carotid artery, and a nerve bundle is stimulated by the electrodes. However, since the nerve bundle in the vicinity of the carotid artery includes various nerve networks, stimuli are given to nerves other than the target nerve, causing considerable side effects. Furthermore, embedding electrodes is highly invasive, imposing a considerable burden on the patient.

In another type of existing nerve stimulating device (e.g., Japanese Translation of PCT International Application, Publication No. 2009-501046), the main unit of the device is indwelled in the stomach, the stimulating electrodes are indwelled in the pancreatic duct, and with the main unit and the stimulating electrodes connected to each other via a lead wire, the entire system is temporarily indwelled in the stomach (main unit), the duodenum (sensor), and the pancreas (electrodes). However, in order to reduce the burden on the patient, it is necessary to make the device as small as possible.

Furthermore, there have been reports that using these existing nerve stimulating devices to increase insulin secretion by stimulating the vagus nerve connected to the pancreas and pancreatic β cells deteriorates the function of the pancreatic β cells in the mid to long term, causing exhaustion of insulin secretion.

Furthermore, nerve networks become thinner and thinner as they go to their distal ends, and from a procedural viewpoint, visual confirmation is very difficult, making it very difficult to find the target nerve network buried in fat or tissue. Furthermore, even if the abdominal cavity is approached surgically, it is difficult to stably indwell electrodes for a nerve network reaching the pancreas or the liver since these organs are located deep inside the body.

In contrast, in the case of the nerve stimulating device 10 according to this embodiment, with the stent 11 indwelled inside the body, the receiving coil 12 receives energy from outside the body, and the stimulating electrodes 13 convert the received energy into electric power at the timing at which the receiving coil 12 receives the energy and passively output the electric power as nerve stimulating pulses.

More specifically, it is possible to promote insulin secretion by stimulating an efferent vagus nerve and pancreatic β cells (insulin-secreting cells) in the vicinity of a bile duct with the nerve stimulating device 10 according to this embodiment indwelled in the bile duct. Furthermore, by stimulating an afferent vagus nerve in the vicinity of a bile duct with the nerve stimulating device 10 according to this embodiment, it is possible to increase the number of pancreatic β cells, thereby increasing insulin secretion.

In this case, with the nerve stimulating device 10 according to this embodiment, it is possible to wirelessly receive energy from outside the body, convert the energy into electric power, and stimulate nerves. In this way, a lead wire for connecting the stent 11 inside the body and a power feeding unit outside the body, as well as a penetrating region where the lead wire penetrates, can be eliminated. This serves to prevent unwanted bacteria, etc. from entering the body from the penetrating region on the body surface, serving to reduce the risk of infection. Furthermore, there is no concern about interruption of the power supply, so that long-term treatment can be provided in a minimally invasive fashion.

Furthermore, with the retaining unit (stent 11) that is guided out from the distal end of the inserted portion of an endoscope and that retains the main unit of the device at a designated position inside the body, it is possible to guide out the nerve stimulating device 10 according to this embodiment with the inserted portion of the endoscope inserted into a body cavity from the distal end (channel) thereof and to place it at the designated position in the body cavity. Thus, it is possible to retain the main unit at the designated position inside the body by using the retaining unit. Accordingly, it is possible to indwell the nerve stimulating device 10 according to this embodiment at the desired position inside the body cavity without performing surgery. This serves to reduce the level of invasiveness, thereby alleviating the burden on the patient.

Furthermore, by constructing the above retaining unit in the form of a stent that can be inserted into a lumen, when guiding out the nerve stimulating device 10 according to this embodiment from the inserted portion of the endoscope, it is possible to contract the stent 11, making the main unit compact. This facilitates the task of guiding out the nerve stimulating device 10. Furthermore, since the stent 11 is a cylindrical component that can be expanded inside the body, it is possible to prevent interference with the flows of body fluids when the stent 11 is indwelled in a body cavity.

Furthermore, by retaining the stent 11 in the vicinity of the gastric cardia of the esophagus, in the duodenum, or in a bile duct, close to where the vagus nerve runs, it is possible to effectively stimulate the vagus nerve running in the vicinity of the gastric cardia of the esophagus or in the vicinity of the duodenum or the bile duct, serving to increase insulin secretion from the pancreas.

Furthermore, by causing the stimulating electrodes 13 to stimulate the vagus nerve distal to the celiac ganglia, it is possible to stimulate only the target nerve. This serves to effectively increase insulin secretion from the pancreas while preventing side effects caused by stimulating other nerves. Furthermore, by stimulating an afferent vagus nerve, it is possible to increase the number of pancreatic β cells while preventing deterioration of the function of the pancreatic β cells in the mid to long term. This serves to effectively increase insulin secretion from the pancreas.

In this embodiment, the stent 11 may be composed of a biodegradable material. More specifically, at least a portion of the stent 11 that is indwelled in a tubular digestive tract is composed of a biodegradable plastic whose main component is, for example, polylactic acid, and the biodegradation speed is controlled so that the stent 11 will lose the retaining force that enables it to be indwelled in the tubular digestive tract and will be detached once the required period of the nerve stimulating treatment elapses and degradation progresses.

When the stent 11 is constructed as described above, after the main unit of the device is indwelled inside the body with the stent 11 for a certain period, the stent 11 loses its retaining force, is detached, and is finally discharged outside of the body. In this way, it is possible to discharge the main unit outside of the body after a certain period instead of permanently indwelling it inside the body, so that an operation for removing the main unit is not needed. Furthermore, in the case of temporary indwelling, it is possible to reduce the retaining force of the stent 11, facilitating its removal from a bile duct.

### Second Embodiment

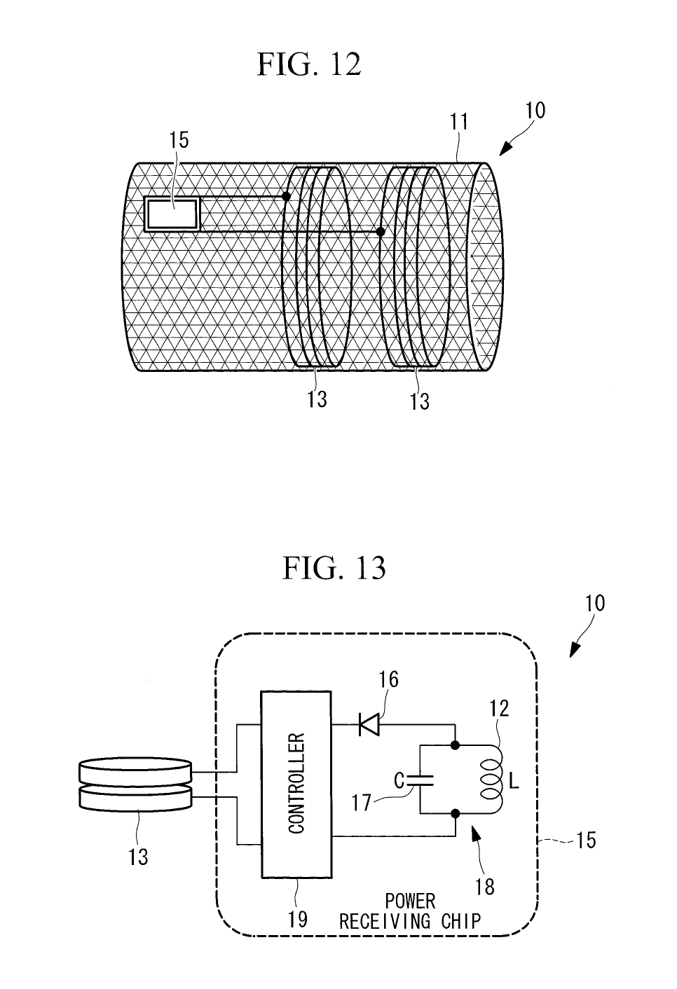

A nerve stimulating system 2 according to a second embodiment will be described below with reference to FIGS. 12 to 16. In the following description, regarding nerve stimulating systems according to individual embodiments, points in common with the nerve stimulating system according to the embodiment described earlier will be designated by the same reference signs, and descriptions thereof will be omitted. The description will be directed mainly to points that differ from the nerve stimulating system according to the embodiment described earlier.

In the nerve stimulating system 2 according to this embodiment, the nerve stimulating device 10 includes a cylindrical stent (main unit, retaining unit) 11, a power receiving chip (receiving unit) 15 provided in the stent 11, and stimulating electrodes (stimulating-pulse outputting unit) 13 provided in the stent 11, as shown in FIG. 12.

The stimulating electrodes 13 are metallic coils disposed along the circumferential direction of the cylindrical stent 11. Multiple stimulating electrodes 13 are disposed in the axial direction (longitudinal direction) of the stent 11, which are electrically connected to the power receiving chip 15.

The stimulating electrodes 13 configured as described above convert energy received by the power receiving chip 15 into electric power at the timing at which the power receiving chip 15 receives the energy from the energy supplying unit 20 placed outside the body and passively output the electric power as nerve stimulating pulses.

As shown in FIG. 13, the power receiving chip 15 includes a receiving coil (receiving unit) 12 that receives AC electric power, a capacitor 17 that stores the AC electric power received by the receiving coil 12, a diode (rectifying unit) 16 that rectifies the AC electric power received by the receiving coil 12, a controller 19 that smoothes the waveform of the electric power rectified by the diode 16 to convert the electric power into DC pulses, and stimulating electrodes (stimulating-pulse outputting unit) 13 that passively output the DC pulses from the controller 19 as nerve stimulating pulses.

The receiving coil 12 and the capacitor 17 are connected in parallel to each other, constituting a secondary-side resonant circuit (LC circuit) 18. The controller 19 may be configured so that the polarities of electric power applied to the stimulating electrodes 13 can be reversed by using an external wireless signal.

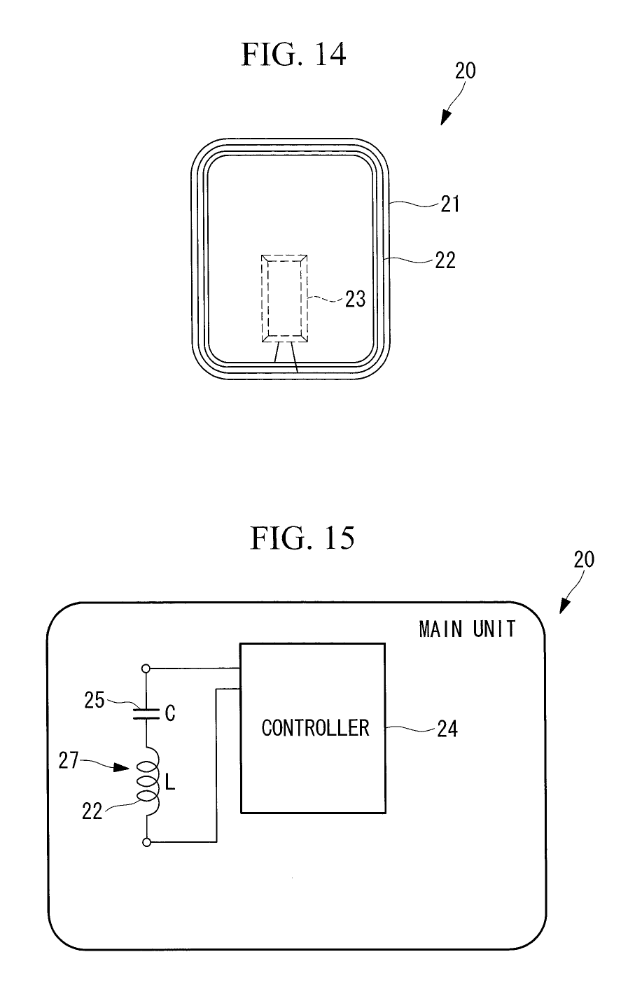

As shown in FIG. 14, the energy supplying unit 20 includes an adhesive sheet 21 that is attached to the body surface, a coil antenna 22 that feeds electric power to the receiving coil 12 of the nerve stimulating device 10, and a generator 23 that supplies electric power to the coil antenna 22.

Furthermore, as shown in FIG. 15, the energy supplying unit 20 includes the coil antenna 22 described above, a capacitor 25 connected in series with the coil antenna 22, and a controller 24 that controls the generator 23 on the basis of a wireless communication instruction from the control device 30.

The coil antenna 22 and the capacitor 25 constitute a primary-side resonant circuit (LC circuit) 27.

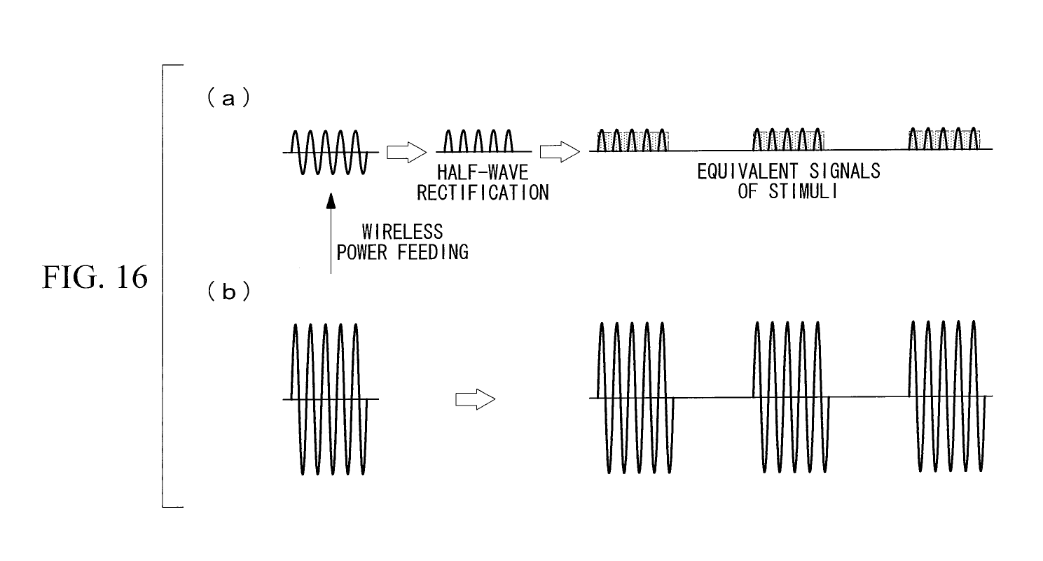

As shown in part (b) of FIG. 16, the controller 24 controls the generator 23 so that AC electric power supplied to the coil antenna 22 is output by causing the primary-side resonant circuit 27 to resonate.

The energy supplying unit 20 configured as described above supplies energy to the nerve stimulating device 10 through magnetic resonance between the primary-side resonant circuit 27 of the energy supplying unit 20 and the secondary-side resonant circuit 18 of the nerve stimulating device 10, for example, at a resonant frequency of 100 kHz. The configuration may be such that a wireless signal can be sent from the energy supplying unit 20 to the controller 19 by a wireless communication unit (not shown) to reverse the polarities of electric power applied to the stimulating electrodes 13. With this configuration, it is possible to choose efferent stimuli and afferent stimuli depending on the case.

With the configuration described above, the receiving coil 12 of the nerve stimulating device 10 receives AC electric power from the energy supplying unit 20 through magnetic resonance. Then, as shown in part (a) of FIG. 16, the AC electric power received by the receiving coil 12 is rectified by the diode 16 (half-wave rectification). The waveform of the rectified electric power is smoothed and converted into DC pulses by the controller 19, which are output from the stimulating electrodes 13 as nerve stimulating pulses.

As described above, with the nerve stimulating system 2 according to this embodiment, the AC electric power received by the receiving coil 12 is rectified by the diode 16, and the rectified electric power is output as pulses (nerve stimulating pulses). This serves to promote insulin secretion from the pancreas.

Furthermore, by causing the primary-side resonant circuit 27 and the secondary-side resonant circuit 18 to resonate with the AC electric power, it is possible to stimulate a nerve with stimulating pulses having an amplified amplitude (i.e., output). This serves to effectively promote insulin secretion from the pancreas.

Furthermore, since these resonant circuits are provided, the effects of external magnetic fields other than those having the resonant frequency are avoided. This serves to prevent incorrect operation due to noise, etc. from the internal circuits and the generation of stimulating pulses from external noise, etc. coming from the environment, which serves to improve the safety of nerve stimulation.

### Third Embodiment

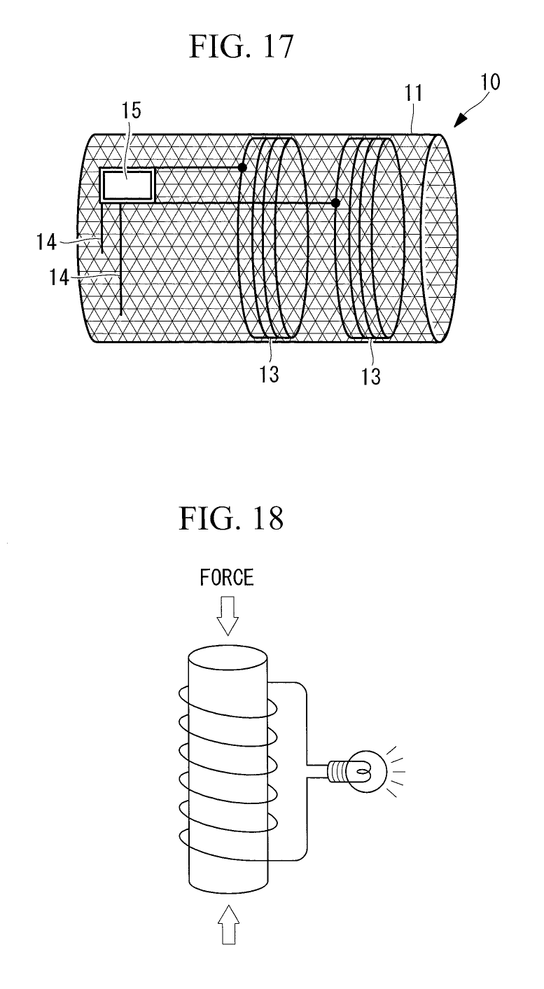

A nerve stimulating system 3 according to a third embodiment will be described below with reference to FIGS. 17 to 19.

In the nerve stimulating system 3 according to this embodiment the nerve stimulating device 10 includes a cylindrical stent (main unit, retaining unit) 11, a power receiving chip (receiving unit) 15 provided in the stent 11, stimulating electrodes (stimulating-pulse outputting unit) 13 provided in the stent 11, and lead wires 14 that electrically interconnect the stent 11 and the power receiving chip 15, as shown in FIG. 17.

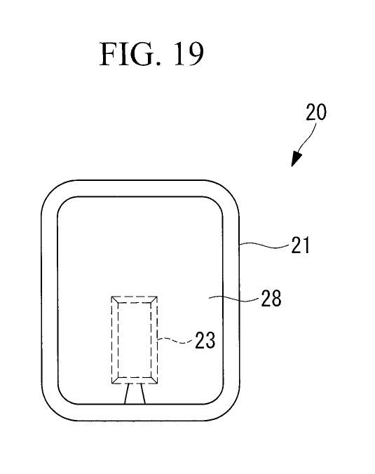

In this embodiment, the stent 11 is composed of a magnetostrictive material exhibiting a magnetostrictive effect. Here, the magnetostrictive effect refers to the effect where, when a force is applied to a magnetostrictive component composed of a magnetostrictive material, the magnetic field in the direction of the force changes, whereby a current is generated on a coil wound circumferentially around the magnetostrictive component, as shown in FIG. 18. Thus, the stent 11 is configured to convert a force (vibration) applied from outside into electric power and to supply the electric power to the power receiving chip 15 via the lead wires 14.

The stimulating electrodes 13 are metallic coils disposed along the circumferential direction of the cylindrical stent 11. Multiple stimulating electrodes 13 are disposed in the axial direction (longitudinal direction) of the stent 11, which are electrically connected to the power receiving chip 15.

As shown in FIG. 19, the energy supplying unit 20 includes an adhesive sheet 21 that is attached to the body surface, a vibrating plate 28 that generates vibration, such as ultrasonic vibrations, and a generator 23 that supplies electric power to the vibrating plate 28 to cause vibration.

The stimulating electrodes 13 configured as described above convert vibration into electric power by the magnetostrictive effect of the stent 11 at the timing at which energy (vibration) is received from the vibrating plate 28 of the energy supplying unit 20 placed outside the body and passively output the electric power as nerve stimulating pulses.

As described above, with the nerve stimulating system 3 according to this embodiment, the stent 11 receives vibrations from outside the body, and the stimulating electrodes 13 output electric power generated by the magnetostrictive effect of the stent 11 as nerve stimulating pulses.

In this way, it is possible to alleviate the effects of external noise and magnetic fields associated with peripheral devices. This serves to improve the safety of nerve stimulation.

The embodiments of the present invention have been described above in detail with reference to the drawings. However, the specific configurations are not limited to the embodiments, and design modifications, etc. within the spirit of the present invention are encompassed. For example, the present invention may be embodied in the form of a suitable combination of the above-described embodiments.

What is claimed is:

1. A nerve stimulating device comprising:
a main unit that is indwelled inside a body;
a receiving unit that is provided in the main unit and that receives energy from outside the body; and
a stimulating-pulse outputting unit that is provided in the main unit and that converts the energy received by the receiving unit into electric power at a timing at which the receiving unit receives the energy and passively outputs the electric power as nerve stimulating pulses.

2. A nerve stimulating device according to claim 1, further comprising a retaining unit that is guided out from a distal end of an inserted portion of an endoscope and that retains the main unit at a designated position in the body.

3. A nerve stimulating device according to claim 2, wherein the retaining unit is a stent that is inserted into a lumen.

4. A nerve stimulating device according to claim 2, wherein the retaining unit retains the main unit in a vicinity of the gastric cardia of the esophagus, in the duodenum, or in a bile duct, close to where a vagus nerve runs.

5. A nerve stimulating device according to claim 2, wherein the retaining unit is composed of a biodegradable material.

6. A nerve stimulating device according to claim 1, wherein the stimulating-pulse outputting unit stimulates an afferent vagus nerve that is located distal to the celiac ganglia.

7. A nerve stimulating device according to claim 1, wherein
the receiving unit receives AC electric power, and
the stimulating-pulse outputting unit includes a rectifying unit that rectifies the AC electric power received by the receiving unit and outputs the electric power rectified by the rectifying unit in the form of pulses.

8. A nerve stimulating device according to claim 7, wherein the stimulating-pulse outputting unit includes a resonant circuit that resonates with the AC electric power received by the receiving unit.

9. A nerve stimulating device according to claim 1, further comprising a magnetostrictive component exhibiting a magnetostrictive effect, wherein
the receiving unit receives vibration from outside the body, and
the stimulating-pulse outputting unit outputs electric power generated by the magnetostrictive effect of the magnetostrictive component as nerve stimulating pulses.

10. A nerve stimulating system comprising:
a nerve stimulating device according to claim 1; and
an energy supplying unit that is placed outside the body and that supplies energy to the receiving unit.

11. A nerve stimulating method, comprising:
indwelling stimulating electrodes for stimulating a nerve in a body cavity;
receiving energy from outside a body by the stimulating electrodes;
converting the received energy into electric power at the timing at which the energy is received; and
passively outputting the electric power as nerve stimulating pulses.

12. A nerve stimulating method according to claim 11, wherein an inserted portion of an endoscope is inserted into the body cavity, and the stimulating electrodes are guided out from the distal end of the inserted portion and are indwelled in the body cavity.

13. A nerve stimulating method according to claim 11,
wherein a retaining unit that retains the stimulating electrodes at a designated position in the body is provided, and
the retaining unit is expanded in the body cavity to retain the stimulating electrodes at the designated position in the body cavity.

14. A nerve stimulating method according to claim 13, wherein the retaining unit is a stent that is inserted into a lumen.

15. A nerve stimulating method according to claim 13, the retaining unit retains the stimulating electrodes in a vicinity of the gastric cardia of the esophagus, in the duodenum, or in a bile duct, close to where a vagus nerve runs.

16. A nerve stimulating method according to claim 11, wherein the stimulating electrodes stimulate an afferent vagus nerve that is located distal to the celiac ganglia.
